(19) United States
(12) Patent Application Publication
(10) Pub. No.: US 2024/0279582 A1
(43) Pub. Date: Aug. 22, 2024

(54) Lid With Variable Interlayer Gap

(71) Applicant: Lucid Scientific, Inc., Atlanta, GA (US)

(72) Inventors: Samuel Walker Inman, Atlanta, GA (US); Nicholas Quan, Richmond Hill, GA (US); Tanner Korb, Atlanta, GA (US)

(21) Appl. No.: 18/570,740

(22) PCT Filed: Jun. 13, 2022
(86) PCT No.: PCT/US2022/033211

§ 371 Date: Dec. 15, 2023

Related U.S. Application Data
(60) Provisional application No. 63/210,638, filed on Jun. 15, 2021.

(51) Int. Cl. C12M 1/00; B01L 3/00; C12M 1/32; G01N 21/77

(52) U.S. Cl. CPC C12M 23/38; B01L 3/50853; C12M 23/12; G01N 21/7703; B01L 2200/025; B01L 2200/141; B01L 2200/142; B01L 2300/0663; B01L 2300/0829; G01N 2021/772

(57) ABSTRACT

A lid comprises a ceiling layer and a floor layer separated by a gap. The ceiling layer is configured to permit application of a force to a control surface in the floor layer. Application of this force adjusts the gap.

### CROSS REFERENCE TO RELATED APPLICATIONS

This application claims priority to U.S. Application No. 63/210,638, filed on Jun. 15, 2021, the contents of which are hereby incorporated by reference in their entirety.

### BACKGROUND

Measuring the rate of oxygen consumed by cells can provide useful information. Cells of interest are often cultured in liquid that is held in a well or receptacle. Multiple wells are often arranged in arrays on a plate. There are numerous formats for these multi-well plates.

To impede entry of contaminants into the wells, it is known to cover the wells with a lid. The lid has a surface configured to cover the wells of a receptacle. In addition to isolating the wells from contaminants, such a lid also maintains humidity and limits evaporation.

A method for carrying out oxygen-consumption measurements is to insert an optical probe into the fluid in which cells are cultured. By moving the probe vertically in the liquid, it becomes possible to measure an oxygen gradient within the well. This oxygen gradient provides a basis for estimating the rate of oxygen consumption.

A system for measuring oxygen consumption rates can include a set of oxygen probes embedded in a lid. Each probe extends substantially perpendicularly from the surface of the lid. The probe is coated at the lower end with a material that is sensitive to a molecule of interest. The material changes optical properties depending on the presence of the molecule of interest. The probe further creates an optical path across the length of the probe and provides an optical interface at the top side of the probe. Movement of the lid relative to the receptacle thus corresponds to movement of the probes in the liquid. Precise probe movement relative to the wells is important for obtaining representative measurements.

As cells consume oxygen, they deplete the supply of oxygen in their immediate vicinity. To avoid cessation of cellular respiration, it is important to transport oxygen from the surrounding environment to replace that consumed. Oxygen transport occurs readily through air surrounding the wells, and less readily within the liquid of a well. Gaps between the lid and wells ensure that oxygen transport in the air above the wells does not limit consumption of oxygen by the cells.

It is also important that wells containing cells remain sterile and that they do not receive contaminants from the surrounding environment. The air path connecting the air directly above the wells to the surrounding atmosphere is often made tortuous such that there are no straight-line paths between these two spaces. A skirt around the perimeter of the lid is often employed for creating this tortuous path.

A lid thus configured covers the wells while maintaining a gas path between the wells and an environment outside the apparatus. It does so without forming any straight-line path that connects a well to the environment. This permits the environment to provide a reservoir to replenish the molecule as it is consumed while still discouraging contamination. In the case of cellular respiration, the molecule in question is an oxygen molecule.

A lid that provides precise placement and movement of probes relative to a well or wells, also allows oxygen transport from the atmosphere, maintains relative humidity levels, and minimizes entry of potential contaminants is an important component of a system for measuring the rate at which cells consume oxygen.

When culturing cells, it is common to use a well plate with an array of wells formed therein. The lower portion of each well holds a liquid that contains a cell culture. The liquid also contains oxygen for use by the cells during respiration. As cellular respiration takes place, a vertical gradient of oxygen concentration forms in this liquid. At any instant, this gradient of oxygen concentration corresponds to the rate of cellular respiration.

An apparatus for measuring oxygen metabolism typically includes a corresponding array of optical probes, each of which extends into a well. The gradient of this oxygen concentration provides information on cellular metabolism. One such apparatus is described in Inman et al., U.S. Patent Publ. 2020/0116600, the contents of which are incorporated herein by reference.

An apparatus for such measurements can include a vertically-oscillating lid. A non-obvious side effect of a moving lid is the change in air volume above the wells. The air drawn inwards along the plane defined by the lid during the upward stroke introduces air from the surrounding environment into the system. This increases the opportunity for contaminants to reach the wells. The exchange of air above the wells also increases the rate of evaporation from liquid in the wells. It is therefore desirable to provide a lid that minimizes the flow of air over the wells relative to that which may occur using a conventional lid.

### SUMMARY

In one aspect, the invention features a lid having a ceiling layer and a floor layer separated by a gap. The ceiling layer is configured to permit application of a first force that pushes against a control surface that transfers the first force to the floor layer. In addition, the ceiling layer is further configured to be held by a second force that constrains its motion. As a result, the ceiling layer moves independently of the floor layer. A change in the first and second forces initiates an adjustment of the gap between the floor and ceiling layers.

Embodiments include those in which the control surface supports the weight of a device that is attached to the ceiling layer. A suitable device is a hand-held device.

Further embodiments include those in which the control surface is not contiguous but is instead the union of two or more discontinuous surfaces that cooperate to support the device. Moreover, the act of pushing against a control surface need not amount to applying a force on each and every point of the control surface.

In some embodiments, the ceiling layer includes an opening disposed above the control surface.

Other embodiments include a probe affixed to the ceiling layer and wherein adjustment of the gap controls an extent to which the probe protrudes from a surface on the bottom of the floor layer or to which the probe protrudes beneath the lid.

Also among the embodiments are those that include a hole in the floor layer.

Still other embodiments include a feature that is biased to exert a force that resists a change in the gap. Suitable features include a flexible element, a spring, a flexure, a set of permanent magnets, or electromagnets, among other features. Among these are embodiments in which the flexible element is biased to exert a force that increases the gap, those in which it is biased to exert a force that resists a change in the gap, and those in which the flexible element and those in which the flexible element, set of flexible elements, or set of magnets are configured such that the gap will to completely collapse in response to a weight of a device that is coupled to the ceiling layer.

Further embodiments include those in which the apparatus includes a feature that exerts a force that is oriented to extend said gap, those in which a feature exerts a force that is oriented to reduce the gap, and those in which a feature exerts a force that restores the gap to an equilibrium position thereof.

Among these are embodiments in which the force is insufficient to prevent the collapse of the gap to a minimum extent thereof in response to a weight that bears down on said ceiling layer when said lid is configured for operation. In some of these embodiments, the gap's minimum extent is zero and, in others, the gap's minimum extent is greater than zero.

In still other embodiments, the ceiling layer comprises an alignment feature to engage a corresponding alignment feature on a device that is to be attached to the ceiling layer.

Still other embodiments are those in which the ceiling layer comprises a coupling structure to securely attach a device thereto. Examples of a suitable coupling structure include a magnet embedded in the ceiling layer, a tab that engages to the device, a groove into which the device slides, or either hooks or loops of a hook-and-loop fastener.

Some embodiments further include a probe affixed to the ceiling layer. In such embodiments, the floor layer includes an opening and adjustment of the gap controls an extent to which the probe extends through an opening in the lid's floor layer.

Also, among the embodiments are those that include first and second extrusions that protrude into the gap from opposite directions. In such embodiments, the first extrusion surrounds the opening in the floor layer. The first and second extrusions form a telescopic arrangement that restricts passage of contaminants through the opening.

Still other embodiments include first and second extrusions that protrude into the gap from opposite directions. In such embodiments, the floor layer comprises an opening and the first extrusion forms a tube that surrounds the opening. In such embodiments, when the lid is in operation, one of said first and second extrusions fits inside the other, thus forming a telescopic structure.

Embodiments include those in which the control surface is disposed on a surface of a post that extends from the floor layer into the gap and towards the ceiling layer and those in which the control surface is disposed within a recess formed in a post that extends from the floor layer into the gap and towards the ceiling layer.

In some embodiments, the floor layer has a bottom surface that can rest on a well or a receptacle containing a well. This bottom surface defines a lid plane The ceiling layer includes an array of probes extending through the gap and beyond the lid plane. The tips of these probes define a probe plane that is parallel to a lid plane. In these embodiments, adjustment of the gap causes vertical translation of the probe plane relative to the lid plane.

Also, among the embodiments are those that further include a vertically-extending member affixed to the ceiling layer and protruding by a variable distance below the lid plane, the distance being dependent on the gap. Among these are embodiments in which vertically-extending member is a probe, those in which it is a pipe, those in which it is a light pipe or optical waveguide, those in which the vertically-extending member provides an optical stimulation, those in which the vertically-extending member provides an electrical stimulation, those in which it is an electrical probe, and those in which it is a liquid guide that guides flow of a liquid into a well for dosing. Among the embodiments are those in which the vertically-extending member is a probe that includes a probe material that emits light in response to oxygen partial pressure.

In still other embodiments, the floor layer is configured with alignment features to engage a particular well plate and wherein the ceiling layer lacks features to engage the particular well plate.

In other embodiments, the floor layer is one of a plurality of floor layers, each of which is configured with alignment features to engage different well plates. In such embodiments, the ceiling layer is configured to operate with each of the floor layers.

In another aspect, the invention features a lid that can vertically oscillate. It achieves this by having a ceiling layer and a floor layer that together define a gap. The floor layer remains stationary on a well plate while the ceiling layer moves vertically relative to the floor layer, thus varying the gap. Perforations in the floor layer enable probes extending from the ceiling layer to enter the wells. Air exchange resulting from movement of the ceiling layer is directed horizontally outwards rather than through the perforations in the floor layer. As a result, the floor layer limits gas exchange between the wells and the environment during movement of the ceiling layer.

In yet another aspect, the invention features an apparatus configured for use when measuring oxygen concentration within each well of an array of wells, each of which can hold a sample that metabolizes oxygen. An example of a sample is a cell culture including a cell that metabolizes oxygen. Such an apparatus includes a multi-layer lid that covers the wells. The layers of the lid have a gap formed therein, and the probes extend through the gap towards the wells.

The apparatus can also include a device. Such a device comprises circuitry for controlling the extent of the gap. Among these are embodiments in which the device comprises an actuator that controls the extent of the gap. Also among these are embodiments in which the device comprises circuitry for processing measurements.

In some embodiments, the lid includes a first layer and a second layer. In these embodiments, the gap is between the first and second layers. The second layer moves relative to the fixed first layer to vary the gap's extent. Among these are embodiments in which the first layer is a floor layer, and the second layer is a ceiling layer. Also, among these embodiments are those in which probes are attached to the ceiling layer.

Among the embodiments are those in which the probes are optical probes that extend through the gap and towards the well. Also among the embodiments are those in which the probe is an electrical probe that extends through the gap towards the well. In such embodiments, changing the extent of the gap causes the probes to move within the well. In some embodiments, increasing an extent of the gap causes the probe's height above the well bottom to increase.

Among the embodiments are those in which the vertically-extending members provide a stimulus in the well. In such embodiments, changing the extent of the gap causes the stimulus to move within the well. In this way, the stimulus can be made to move closer to or farther from the sample in order to increase or decrease the intensity of the stimulus relative to the sample.

Among the embodiments are those that include a device removably attached to the lid. The device includes a circuitry to control the measurement process. The device can also include a plurality of optoelectronic sensors arranged to acquire optical signals from probes immersed within a fluid that is contained within the wells.

Among the embodiments are those in which the ceiling layer of the lid is fixedly attached to the device. As a result of such attachment, the optoelectronic sensors in the device maintain alignment with the probes embedded in the ceiling layer. In some of these embodiments, the device is fastened, attached, or placed so as to resist movement relative to the lid or to the probes. Among these are embodiments in which the attachment thus achieved, while being fixed, is not permanent. A suitable way to achieve such fixed but impermanent attachment is to use one or more magnets to attach the device.

The device can further include an actuator. The actuator can comprise an adjustment screw coupled with a motor. The motor rotates the adjustment screw which, as a result of interacting with the floor layer, controls the extent of the gap. Embodiments include those with more than one such adjustment screw.

Still other embodiments include a spring. Among these are embodiments in which the spring is biased to change the extent of the gap and embodiments in which the gap is biased to expand when the device is not present. Properties of the spring can be set such that the weight of the device that it supports induces the gap to collapse entirely.

In still other embodiments, the lid's floor layer includes holes, each of which corresponds to one of the probes. Each hole permits a probe to pass through the floor layer and into its respective well.

Among the embodiments are also those that include a motion arrestor that limits variation in the gap. Among these are embodiments that include first and second motion-arrestors. The first motion-arrestor limits a minimum extent of the interlayer gap and the second motion-arrestor limits a maximum extent of the gap. In some embodiments, a motion arrestor takes the form of a post.

In some embodiments, the floor layer further includes a post formed integrally therewith. The post extends into a control opening in the ceiling layer. The post can provide a platform for the adjustment screw such that extension of the adjustment screw raises the device. In some embodiments, the adjustment screw also raises the ceiling layer, thus extending the gap between the layers. Each post has a vertical extent that depends on the extent to which the probe is to penetrate into the well. In some embodiments, the opening includes an impediment that prevents the post from being removed from the opening.

Among the embodiments are those in which the floor layer includes a post surface and the actuator, which is within the device, includes a screw that extends downward towards the post surface through openings in the ceiling layer. Rotation of the screw controls vertical travel of the ceiling layer.

In other embodiments, the lid includes a floor and ceiling layer with the floor layer's structure depending on that of a well plate that contains the wells and the ceiling layer's structure being agnostic to the well plate.

In some embodiments, the floor layer includes motion-arresting features and features that interface the adjustment screws. Such floor-layer features can be set to correspond with different versions of well plates such that the position of the probes within the wells of a well plate remain relatively constant for identical device and ceiling layer configurations despite differences in well plates.

In still other embodiments, the lid includes first and second layers, at least one of which was created by a process selected from the group consisting of a subtractive manufacturing process, an additive manufacturing process, and an injection molding process.

Among the embodiments are those in which the lid includes a floor layer that covers the array of wells and a ceiling layer arranged at a variable distance above the floor layer thereby creating the gap therebetween. In such embodiments, the actuator is configured to cause the ceiling layer to move, thereby causing an extent of the gap to vary. Among these are embodiments in which the gap's extent varies between minimum and maximum values.

Among the foregoing embodiments are those in which the floor layer includes holes to permit the probes to pass therethrough and enter corresponding wells.

Also among the foregoing embodiments are those in which the ceiling layer includes a flexible element integrally formed therewith. In such embodiments, the flexible element is biased to exert a force that urges the ceiling and floor layers apart. Alternatively, the flexible element can be included in the floor layer. In some embodiments, the flexible element is a spring.

In some embodiments, extrusions prevent a straight-line path between the surrounding environment and a hole in the floor layer. Embodiments with extrusions also include those in which the extrusions are integrated within the floor layer, within the ceiling layer, or within both the floor and ceiling layers. Also, among the embodiments having one or more extrusions are those in which an extrusion defines an area around the hole in the floor layer. The product of this area and the variation in the gap governs the volume of gas that will be exchanged based on the variation of the gap. The extrusion can be designed such that this area is minimized so as to match the area of the hole. Among these embodiments are those in which the area defined by an extrusion surrounding a hole in the floor layer is less than sixty-four square millimeters, those in which it is less than thirty-two square millimeters, and those in which it is less than sixteen square millimeters. Embodiments also include those in which each extrusion defines an area around only one hole in the floor layer and those in which each hole in the floor layer is surrounded by its own extrusion.

In other embodiments, the two layers are constrained to be together. Among these embodiments are those in which the floor layer further includes a post formed integrally therewith. The post extends through an opening in the ceiling layer and has proximal and distal sections. The distal section's cross-sectional area is greater than that of the proximal section. An axial extent of the proximal section sets a maximum extent of the interlayer gap. Such embodiments are distinguished over an alternative embodiment in which the layers are permitted to separate.

In still other embodiments, the ceiling layer includes an opening. The floor layer includes a post surface that is accessible through the opening. The elevation of the post surface together with the length of the probe and the interlayer gap sets the amount a probe material extends through the floor layer.

Some embodiments include a mating feature that extends from a bottom surface of the floor layer and aligns with one or more of the wells.

Also among the embodiments are those that achieve alignment of probes in the lid with wells in the receptacle using bumps on the inside surface of the vertical portion of the lid. These surfaces interface with tapered outside walls of the well plate. Among these embodiments are those in which the bumps are configured as flexures. Such embodiments provide flexibility to accommodate variations in the geometry of the tapered walls.

In another aspect, the invention features an apparatus that includes a lid that comprises a ceiling layer and a floor layer separated by a gap. The lid includes means for receiving a first force that constrains motion of the ceiling layer such that the ceiling layer moves independently of the floor layer and means for permitting application of a second force to a control surface inside the lid. A change in the first and second forces initiates an adjustment of an extent of the gap.

### DESCRIPTION OF DRAWINGS

FIG. 1 shows an isometric view of a lid;

FIG. 2 shows an exploded isometric view of the lid of FIG. 1;

FIG. 3 shows an isometric view of a well plate to be covered by the lid of FIG. 1;

FIG. 4 shows an exploded side view of a lid above a well plate with a device on top of the lid;

FIGS. 5-7 shows sectional views of a lid above a well plate in several positions;

FIG. 8 is an isometric view of the top side of the floor layer of the lid of FIG. 1;

FIG. 9 is an isometric view of the bottom side of the floor layer of the lid of FIG. 1;

FIG. 10 is an isometric view of the top side of the ceiling layer of the lid of FIG. 1;

FIG. 11 is an isometric view of the bottom side of the ceiling layer of the lid of FIG. 1;

FIG. 12 is a section view showing different adaptations of the floor layer of the lid; and

FIG. 13 shows two positions of a partial cross section of the lid coupled with the device of FIG. 4 and resting on top of the well plate of FIG. 4.

### DESCRIPTION

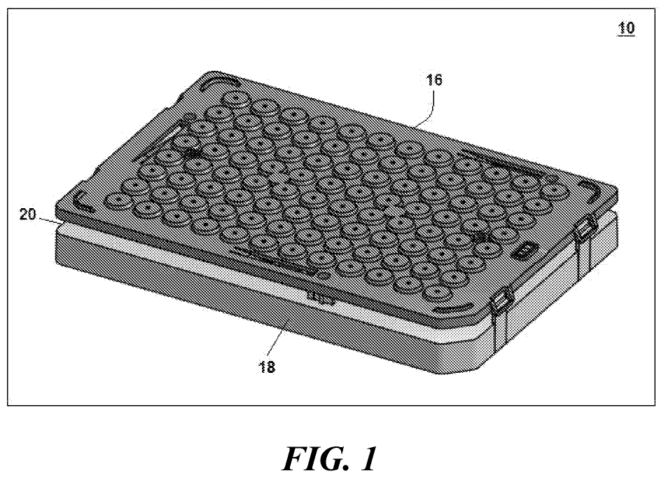

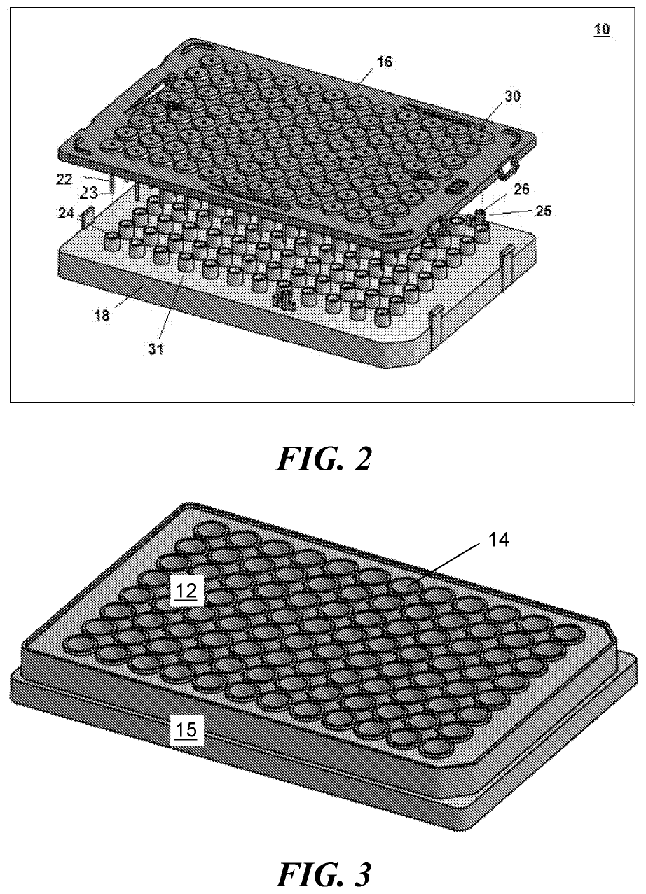

FIGS. 1 and 2 show a lid 10 that has a ceiling layer 16 separated from a floor layer 18 by an interlayer gap 20. The ceiling layer 16 defines a transverse plane and a vertical direction that is perpendicular to the transverse plane. As shown herein, the lid comprises at least the ceiling layer 16 and a floor layer 18. However, in some embodiments, one or more additional layers exist between the ceiling and the floor layer 18, below the floor layer 18, or above the ceiling layer 16.

A variety of ways are available for forming the ceiling layer 16 or the floor layer 18. These include injection molding, the use of an additive manufacturing process, and the use of a subtractive manufacturing process. In some embodiments, the floor and ceiling layers 16, 18 are formed using different manufacturing processes.

A probe 22 fixed to the ceiling layer 16 extends downward towards the floor layer 18. As a result of its alignment with a corresponding hole 24 in the floor layer 18, the probe 22 passes freely through the floor layer 18. As shown in FIG. 2, the probe 22 is one of a plurality of probes, each of which passes through a corresponding hole 24 in the floor layer 18.

Each probe 22 has a distal end on which is affixed a probe material 23 that is sensitive to a chemical of interest. As a result, the probe 22 is able to provide information concerning the chemical of interest in the environment that surrounds the probe's distal tip.

When in use, the lid 10 covers a well plate 15, as shown in FIG. 3. The well plate 15 contains an array 12 of wells 14. Among these wells 14 are those that hold a sample whose consumption of oxygen is of interest. For such an application, the probe material 23 emits light in response to oxygen partial pressure. In these embodiments, the probe 22 includes an optical fiber or a light pipe or optical waveguide that captures this light and guides it away from the probe's distal tip.

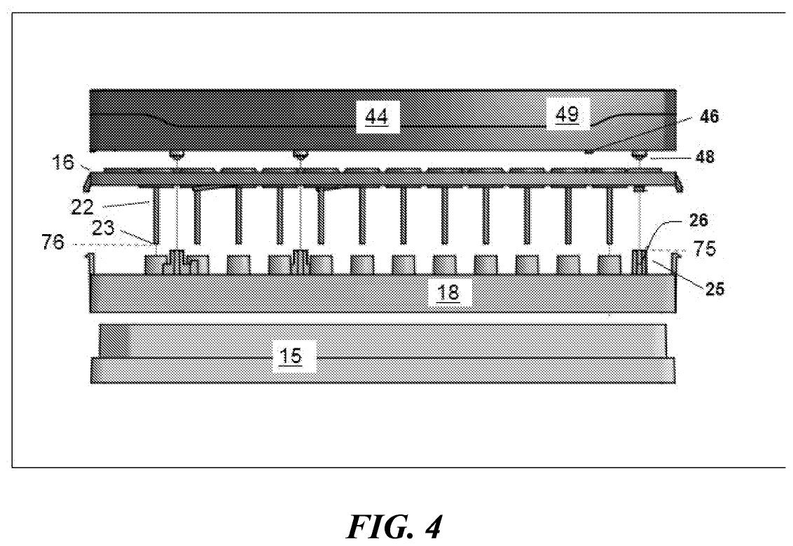

As shown in FIG. 4, the lid 10 supports a device 44 towards which the probe 22 guides the captured light. The device 44, which is separate from the lid 10, comprises electronic circuitry that controls one or both of the measurement process and the movement process.

The lid 10 supports the device 44 on its ceiling layer 16. To promote its ability to maintain alignment with the device 44, the lid 10 features an alignment recess 60 that engages a corresponding alignment feature 46 that extends from the device 44. This alignment recess 60 assists the lid 10 in aligning the device 44 that it supports relative to the ceiling layer 16 of the lid 10.

A particularly useful feature of the lid 10 is its ability to permit adjustment of the interlayer gap 20 from outside of the lid 10. This is achieved by providing an opening in the ceiling layer 16 through which it is possible to impose a force against a control surface 26 that is coupled to the floor layer 18 so that a force applied to the control surface 26 is transferred to the floor layer 26 The ability of the floor layer to support this force provides a way to change the extent of the interlayer gap 20 from outside the lid 10 itself.

In some embodiments, the control surface 26 that bears the force is on the floor layer 18 itself. In other embodiments, the control surface 26 that bears the force is on or in a structure, such a control post 25, that rises from the floor layer 18 so that application of the force to control surface 26 transmits the force to that structure, which then transmits it to the floor layer 18.

By permitting interaction with a surface coupled to its floor layer 18 even through the intervening ceiling layer 16, the lid 10 makes it possible to raise and lower the probes 22 into the wells 14 without compromising the sterility of the wells 14.

The device 44 couples to the ceiling layer 16. As a result, the device 44 constrains motion of the ceiling layer 16. If the device 44 moves relative to the floor layer 18, so too does the ceiling layer 16.

Meanwhile, the ceiling layer 16 is configured so as to permit the floor layer 18 to support the device 44, even though the device 44 is on the ceiling layer 16. In particular, the control surface 26 supports the weight of the device 44.

As a result of this configuration, in which the device 44 both constrains the motion of the ceiling layer 16 and also supports itself on the floor layer 18, it is possible for the device 44 to adjust the interlayer gap 20. For example, to increase the gap 20, the device 44 bears down on the floor layer 18, thus raising itself off the floor layer 18 and taking the ceiling layer 16 with it. Conversely, to reduce the gap 20, the device 44 lowers itself, thus lowering the ceiling layer 16 at the same time.

To raise and lower the probes 22, the lid 10 receives a fine-pitched adjustment screw 48 that is mounted on the device 44. An actuator 49 on the device 44 drives the adjustment screw 48 and controls how far the fine-pitched adjustment screw 48 extends downward. In doing so, the actuator 49 also controls the interlayer gap 20. In a preferred embodiment, the actuator 49 comprises a stepper motor that turns the adjustment screw 48 in either a first or second direction.

Rotating the adjustment screw 48 in a first direction causes it to extend and to thus engage the control surface 26 such that the control surface 26 supports the weight of the device. Since the control surface 26 of the floor layer 18 supports the weight of the device 44, variations in the extent of the adjustment screw 48 induce variations in the space between the device 44 and the floor layer 18. Since the ceiling layer 16 remains in contact with the device 44 throughout these variations, the interlayer gap 20 is adjustable from outside the lid 10. Since the probes 20 are affixed to the ceiling layer 16, the lid 10 also controls vertical movement of the probe material 23.

FIGS. 2 and 4 show a control opening 30 formed in the ceiling layer 16. The adjustment screw 48 shown in FIG. 4 passes through this control opening 30. As a result, the lid's control opening 30 permits the control surface 26 to interact with the device 44 without interference from the ceiling layer 16 that lies between the device 44 and the floor layer 18.

The control opening 30 opens directly above the control post 25, which extends upward from the floor layer 18. An upper surface of the control post 25 serves as a control surface 26 against which the adjustment screw 48 abuts. When the actuator 49 turns the adjustment screw 48 in one direction, the adjustment screw 48 extends outward, thus pushing off against this control surface 26. This simultaneously raises the ceiling layer 16, enlarges the interlayer gap 20, and raises the device 44. When the actuator 49 turns the adjustment screw 48 in the opposite direction, the adjustment screw 49 retracts, thus lowering the ceiling layer 16, shrinking the interlayer gap 20, and lowering the device 44.

In effect, the control surface 26, the adjustment screw 48, and the actuator 49 all cooperate to carry out what, for a human being, would be referred to as a “push-up.” Rotating the adjustment screw 48 in a first direction causes it to extend and to thereby engage the control surface 26, which has a normal vector having a component in the vertical direction. The control surface 26 exerts an upward vertical force to support the device 44 as the ceiling layer 16 is raised, thus increasing the lid's interlayer gap 20. Since the device 44 rests on the ceiling layer 16, the height of the ceiling layer 16 and the extent of the interlayer gap 20 are constrained by the device 44. Since the probes 20 are coupled to the ceiling layer 16, raising and lowering the ceiling layer 16 also raises and lowers the probes 20.

In FIG. 4, the control surface 26 is defined by the union of three surfaces spread across three such control posts 25. These three surfaces define a control plane 75. Similarly, the height of a probe material 23 at the distal tip of a probe 22 defines a probe plane 76. Turning the adjustment screw 48 changes the position of the probe plane 76 relative to the position of the control plane 75.

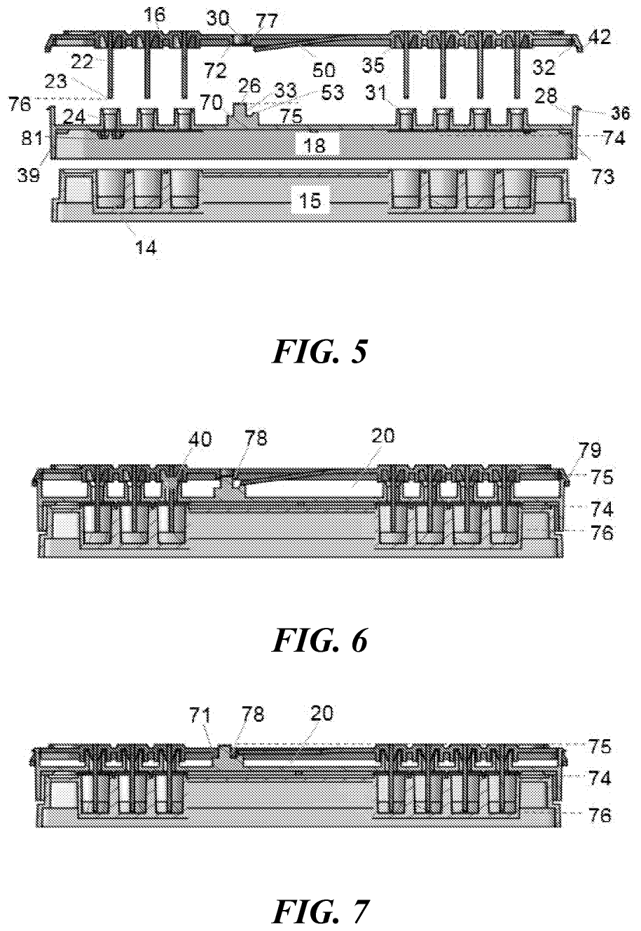

FIG. 5 shows an exploded view of the lid 10 on a well plate 15 with the ceiling layer 16 having been detached from the floor layer 18 and with the device 44 having been separated from the ceiling layer 16.

FIG. 6 shows the lid 10 from FIG. 5 after having been assembled. In FIG. 6, the extent of the gap 20 has set the distance between the probe plane 76 and the control plane 75 such that distal tips of the probes 20 lie above the bottoms of the wells 14 into which they have been inserted.

FIG. 7 shows the lid 10 from FIG. 6 after having turned the adjustment screw 48 shown in FIG. 4 to reduce the interlayer gap 20. This further increases the distance between the probe plane 76 and the control plane 75. As a result, the distal tips of the probes 20 move closer to the bottoms of their respective wells 14.

Referring now to FIGS. 6 and 7, when the lid 10 rests on a well plate 15, a well-plate interface 73 in the lid's floor layer 18 interfaces with the well plate 15. In some embodiments, the well-plate interface 73 includes multiple parts or multiple features that together define a bottom plane 74 of the floor layer 18. When the lid 10 rests on a well plate 15, the position of the floor layer's bottom plane 74 remains constant relative to the well 14 even as variations in the gap 20 vary the position of the probe material 23 within the well 14.

Raising or lowering the ceiling layer 16 controls a probe's vertical movement. In particular, lowering the ceiling layer 16 causes more of the probe 22 to extend through its corresponding hole 24. Raising the ceiling layer 16 causes less of the probe 22 to extend through its corresponding hole 20.

The gap's extent also controls the distance between the probe plane 76 and the floor layer's bottom plane 74. Increasing the gap 20 moves the probe plane 76 towards the floor layer's bottom plane 74 and decreasing the gap 20 moves the probe plane 76 away from the floor layer's bottom plane 74. Consequently, when the floor layer's bottom plane 74 rests on the well plate 15, the distance between the probe material 23 and the bottom of a well 14 depends on the gap's extent.

Raising the ceiling layer 16 pulls the probe 22 away from the bottom of its corresponding well 14. During this process, the floor layer 18 remains fixed on the well plate 15 so that the changes in the gap's extent relate directly to changes in the vertical position of the probe 22 in the well 14. The lid's floor layer 18 continues to cover the wells 14 even as the probes 22 move up or down. Since the floor layer 18 covers the wells 14 the entire time, the wells 14 remain sterile.

As can also be seen in FIG. 6, the floor layer 18 includes a first motion arrestor 79 that limits the extent of the gap 20. The first motion arrestor 79 prevents the lid 10 from being raised further than what is shown in FIG. 6. This prevents the ceiling 16 and the floor 18 of the lid 10 from coming apart.

FIG. 7 shows the lid 10 of FIG. 6 but with the ceiling layer 16 shown in FIG. 7 having been lowered to an extent that minimizes the gap 20 between the ceiling layer 16 and floor layer 18. As a result, this position will be referred to herein as the “collapsed position.” The minimum extent of the gap 20 is limited by a second motion-arrestor 71 between a floor surface 70 of the floor layer 18 and a ceiling surface 72 in the ceiling layer 16.

As shown in FIG. 5, the control surface 26 comprises the top surface of a control post 25 having an alignment surface 33 whose normal vector extends in the transverse direction. The control opening 30 in the ceiling layer 16 has a control-opening wall 77. When the lid 10 is assembled, the vertical alignment surface 33 engages the control-opening wall 77 to form an alignment interface 78, as shown in FIG. 6. This promotes a horizontal orientation of the ceiling layer 16 during raising and lowering thereof.

Some embodiments feature more than one instance of the alignment interface 78. To provide constraint on two degrees-of-freedom, it is particularly useful to have two instances of the alignment interface 78. These instances of the alignment interface 78 cooperate in constraining horizontal translation and rotation of the floor layer 18 and ceiling layer 16 relative to each other, thus promoting the ceiling layer's ability to move in only a vertical direction.

The control post 25 has a cross section that remains relatively constant along its length and that matches the cross-section of the control opening 30. As a result, the control post 25 slides freely into and through the control opening 30.

As seen in FIG. 5, a limit post 28 that extends from the floor layer 18 forms a limit-post ledge 36 having a surface that defines a first normal vector. Meanwhile, a limit opening 32, also seen in FIG. 5, has a stepped profile that forms a limit-opening ledge 42 that defines a second normal vector. Both the first and second normal vectors thus defined have a non-zero vertical component.

When the lid 10 is assembled, a surface of the limit-post 28 lies above a corresponding surface of the limit-opening ledge 42. As a result, the limit-opening ledge 42 and the limit-post ledge 36 engage each other to form the first motion-arrestor 79. The first motion-arrestor 79 limits the maximum extent of the upper lid's vertical movement, thereby setting the maximum gap 20.

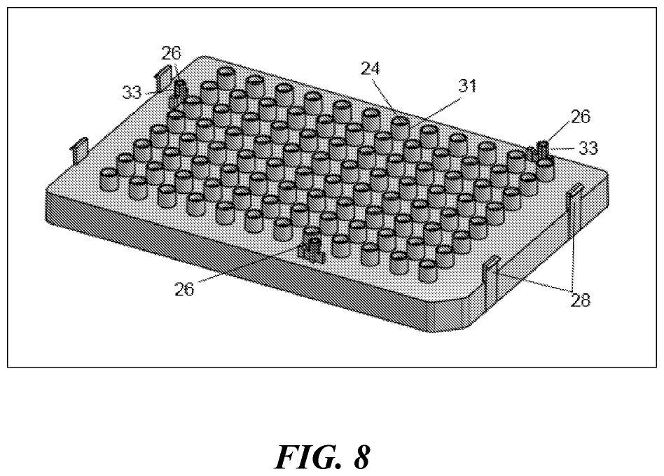

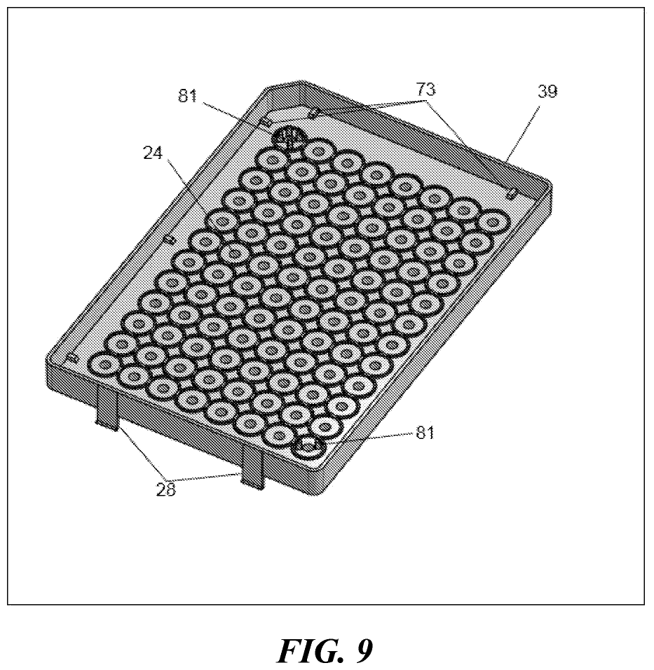

FIG. 8 shows the floor layer 18 as seen from within the gap 20 and FIG. 9 shows the floor layer 18 as seen from within the array 12 that the lid 10 fits over, including a well-plate interface 73.

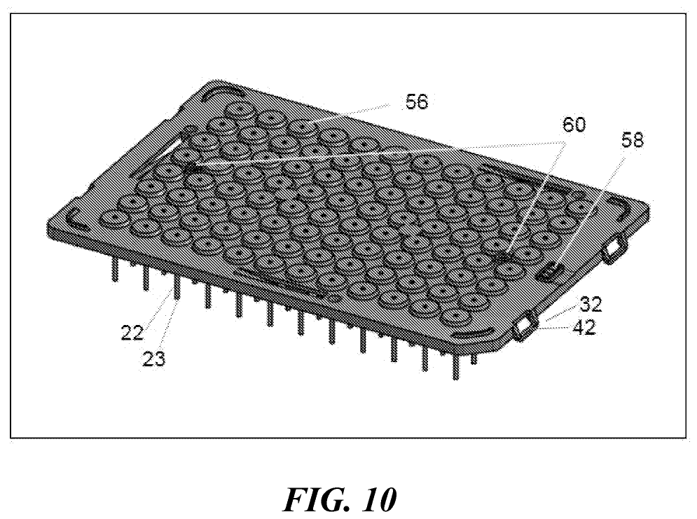

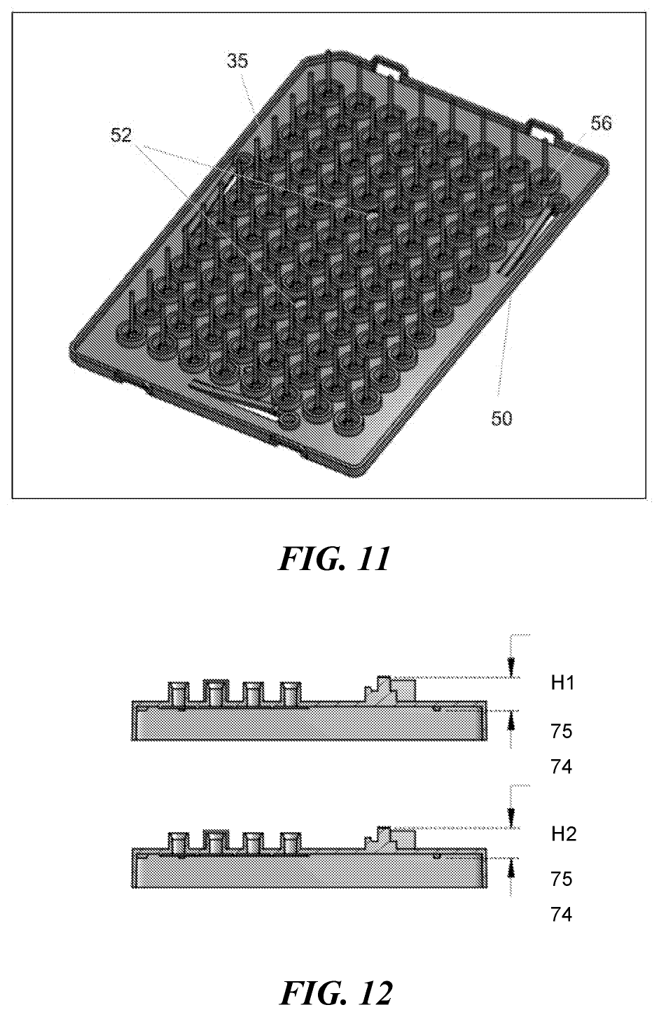

FIG. 10 and FIG. 11 shows the ceiling layer 16, which is intended to be attached to the device 44. It is important that the ceiling layer 16 remain attached to the device 44 while the device 44 lifts itself away from the floor layer 18. A variety of coupling structures are available to securely attach the device 44 to the ceiling layer 16. Among these are magnets 52 embedded in the ceiling layer 16. Alignment features 46 in the device 44 can couple with alignment features 60 in the ceiling layer 16 to promote horizontal alignment of the device 44 and ceiling layer 16.

FIG. 8 also shows a first extrusion 31 that surrounds a probe hole 24 and that extends into the gap 20. FIG. 2 shows the same first extrusion 31 in relation to a ceiling layer 16.

The first extrusion 31 creates a tortuous path between the environment external to the lid 10 and the probe hole 24 that it surrounds. As can be seen in FIG. 11, a second extrusion 35 surrounds the probe 20 that is to enter the probe hole 24. The first and second extrusions 31, 35 thus form a telescoping arrangement that makes it more difficult for contaminants to reach that probe hole 24 and also suppresses movement of air within a well 14 when the interlayer gap 20 is being adjusted. As a result, the first extrusion 31 and the second extrusion 35 can be regarded as defining a “sterility feature.”

As shown in FIG. 9, the floor layer 18 includes a third extrusion 81 that extends downward from the floor layer 18 and into the well 14. The third extrusion 81 promotes horizontal alignment between the floor layer 18 and the well plate 15.

As shown in FIG. 5, a spring 50 integrally formed with the ceiling layer 16 interfaces with a spring seat 53 in the floor layer 18 to bias the extent of the gap 20. This provides a default position of the ceiling layer 16 relative to the floor layer 18. Embodiments include those in which the spring 50 is a flexible element or flexure that is made of the same material as the bulk of the ceiling layer 16 and is formed with the ceiling layer 16 as part of the manufacturing process for the ceiling layer 16. In a preferred embodiment, the spring 50 is biased to expand the gap 20. The spring 50 thus promotes attachment between the device 44 and the ceiling layer 16 as the adjustment screw 48 is adjusted.

To control the extent of the gap 20, the spring's spring constant is preferably adjusted such that the spring 50 does not support the weight of the device 44 over the ceiling layer's range of travel. Ideally, the spring constant of the spring 50 is set such that the weight of the device 44 causes the gap 20 to collapse entirely.

In a preferred embodiment, the device 44 is a handheld device weighing roughly 250 grams. In one embodiment, a one-kilogram mass is sufficient to collapse the gap to its minimum extent. In a preferred embodiment, the weight of a 250-gram mass is sufficient for collapsing the gap 20 to its minimum extent.

In general, well plates 15 made by different manufacturers have different well depths. FIG. 12 shows different adaptations of the floor layer 18 where the distances H1, H2 between the control plane 75 and the bottom plane 74 has been adjusted to accommodate different well plates 15. The adjustments have been made such that placement of the probe material 23 within the well 14 remains comparable across different well designs. As a result, different models of floor layer 18 can be used for interfacing with well plates 15 produced by different manufacturers. However, features of the interface that the floor layer 18 presents to ceiling layer 16 can remain constant. This permits the ceiling layer 16 to remain agnostic to the format of the underlying well plate 15.

In addition to differing well designs, different well plates 15 also have structural differences that relate to proper alignment and seating of a lid 10. Because only the floor layer 18 is specific to a particular well plate 15, these manufacturer-specific details can be accommodated by adaptations in the floor layer 18 alone. This results in another non-obvious advantage. In addition to suppressing air flow above the wells, the lid 10 promotes manufacturing convenience by confining all manufacturing-specific details to the floor layer 18 while retaining a standardized ceiling layer 16 that is compatible with all floor layers 18 and hence with all well plates 15 for which suitable floor layers 18 exist.

Having a well-agnostic ceiling layer 16 is advantageous because the ceiling layer 16 is inherently more complex than the floor layer 18. As is apparent from the isometric view of the ceiling layer 16 shown in FIG. 11, there exist magnets 52 that hold the ceiling layer 16 to the device 44. The ceiling layer 16 also includes anchors 56 that hold a rigidly-attached fiber that forms part of the probe 22. As can be seen in FIG. 10, the ceiling layer 16 also includes circuitry 58 that interfaces with the device 44 and pockets 60 for receiving alignment pins. By isolating all well-specific details to the floor layer 18, it becomes possible to manufacture a universal ceiling layer 16 for functions with different well plates 15.

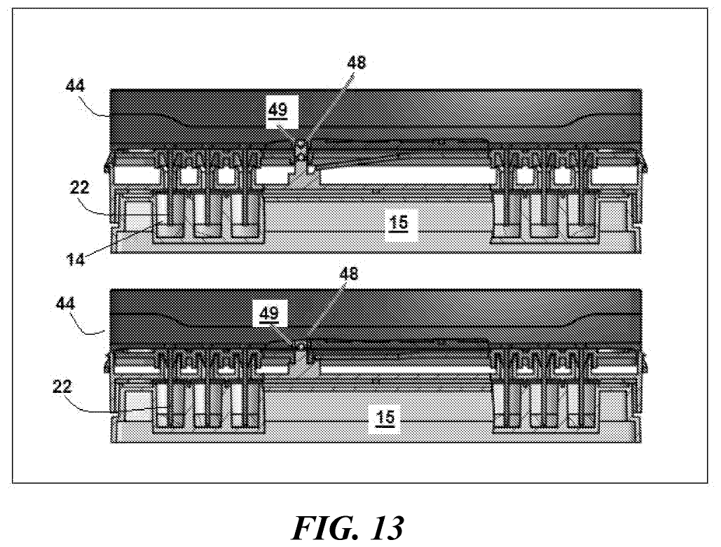

FIG. 13 shows the device 44 with an actuator 49 coupled to the adjustment screw 48. By working against the weight of the device 44, the adjustment screw 48 smoothly varies the extent of the gap 20, and hence the positions of the probes 22 in the wells 14. This permits the probes 22 to move vertically in a controlled manner to measure an oxygen gradient in the well 14.

The floor layer 18 remains over the wells 14 during the entire procedure. As shown in FIG. 9, a wall 39 around the perimeter of the floor layer 18 contributes to the creation of a tortuous path that limits the entry of contaminants into the wells 14 of the well plate 15 by preventing a straight-line path between the environment and the well 14.

The first extrusions 31 and the second extrusions 35, which can be seen in FIGS. 8 and 11, respectively cooperate to form sterility features, that further prevent a straight-line path between the space above the probe hole 24 and the space within the well's interior across the lid's entire range-of-travel.

As the actuator within the device 44 moves the ceiling layer 16, air is either drawn into or expelled from the gap 20. However, because of the sterility features formed by the first and second extrusions 31, 35, this movement of air tends not to disturb the air within the well 14. This minimizes evaporation and the potential for contamination.

In some embodiments, it is possible to use the motion of the ceiling layer 16 as a mechanism for delivering a dose of some liquid additive into the well 14. In particular, additive liquid injected into the gap 20 will tend to pool on the floor layer 18. As the gap 20 is reduced, the probes 22 will pass through such a pool and pick up some of the additive liquid. A probe 22 then enters the well 14 coated with a thin layer of the additive liquid. The additive liquid then drips off the probe 22 and into the well 14. In some embodiments, additional channels provide a pathway for liquid additives into the wells 14 without having to rely on the movement of the probe 22 for delivery.

The ability to manipulate the gap 20 between the floor layer 18 and the ceiling layer 16 is also useful for purposes other than controlling the height of the probes 22. In some embodiments, it is possible to orchestrate the release of one or more fluids by transitioning through a sequence of configurations.

Some embodiments feature one or more latches that can be activated or deactivated to program the apparatus to release liquids on cue. In some of these embodiments, a first configuration either activates or deactivates the latch. A second configuration, which follows the first, releases liquid if the latch has been activated. Otherwise, the second configuration releases no liquid.

Among the embodiments that permit programmatic liquid release are those in which the extent of the gap 20 controls the state of the latch. As an example, in some embodiments, the gap 20 activates the latch by increasing beyond some threshold. This threshold can be set outside the limits of normal operation. Subsequent movements of the lid 10 can ensure the entire volume of fluid has been distributed.

Programmatic release of liquids can be carried out by multiple latches that cooperate to release liquid at different times. Other embodiments feature one latch that is configured to control release of multiple liquid samples.

1. An apparatus comprising a lid that comprises a ceiling layer and a floor layer separated by a gap, wherein said ceiling layer is configured to permit application of a first force that pushes against a control surface that transfers said first force to said floor layer, wherein said ceiling layer is further configured to be held by a second force that constrains motion of said ceiling layer such that said ceiling layer moves independently of said floor layer, wherein a change in said first and second forces initiates an adjustment of said gap.

2. The apparatus of claim 1, further comprising a probe affixed to said ceiling layer and wherein adjustment of said gap controls an extent to which said probe protrudes beneath said lid.

3. The apparatus of claim 1, further comprising a probe affixed to said ceiling layer and wherein adjustment of said gap controls an extent to which said probe protrudes from a surface on the bottom of the floor layer.

4. The apparatus of claim 1, further comprising a hole in said floor layer.

5. The apparatus of claim 1, wherein said ceiling layer comprises an opening disposed above said control surface.

6. The apparatus of claim 1, wherein said ceiling layer comprises a coupling structure to securely attach a device thereto.

7. The apparatus of claim 1, further comprising a magnet embedded in said ceiling layer.

8. The apparatus of claim 1, wherein said ceiling layer comprises an alignment feature to engage a corresponding alignment feature on a device that is to be attached to said ceiling layer.

9. The apparatus of claim 1, further comprising a feature that exerts a force, the force being oriented to extend said gap.

10. The apparatus of claim 1, further comprising a feature that exerts a force that is oriented to extend said gap, said force being insufficient to prevent said gap from collapsing to a minimum extent thereof in response to a weight that bears down on said ceiling layer when said lid is configured for operation.

11. The apparatus of claim 1, further comprising first and second extrusions that protrude into said gap from opposite directions, wherein said floor layer comprises an opening, wherein said first extrusion forms a tube that surrounds said opening, and wherein one of said first and second extrusions fits inside the other.

12. The apparatus of claim 1, wherein said control surface is disposed on a surface of a post that extends from said floor layer into said gap and towards said ceiling layer.

13. The apparatus of claim 1, wherein said control surface is disposed within a recess formed in a post that extends from said floor layer into said gap and towards said ceiling layer.

14. The apparatus of claim 1, wherein said ceiling layer comprises an array of probes extending through said gap and protruding out of said lid, wherein tips of said probes define a probe plane that is parallel to a lid plane defined by said lid, and wherein adjustment of said gap causes vertical translation of said probe plane relative to said lid plane.

15. The apparatus of claim 1, further comprising a vertically-extending member affixed to said ceiling layer and protruding by a variable distance below said floor layer, said distance being dependent on said gap.

16. The apparatus of claim 10, wherein said vertically-extending member comprises an optical waveguide.

17. The apparatus of claim 10, wherein said vertically-extending member comprises a probe.

18. The apparatus of claim 10, vertically-extending member comprises a probe and wherein said probe comprises a probe material that emits light in response to oxygen partial pressure.

19. The apparatus of claim 10, wherein said vertically-extending member comprises a fluid guide that is wetted by a liquid that is to be delivered into a well below said lid.

20. The apparatus of claim 1, wherein said floor layer is configured with alignment features to engage a particular well plate and wherein said ceiling layer lacks features to engage said particular well plate.

21. The apparatus of claim 1, wherein said floor layer is one of a plurality of floor layers, each of which is configured with alignment features to engage different well plates and wherein said ceiling layer is configured to operate with each of said floor layers.

22. An apparatus comprising a lid that comprises a ceiling layer and a floor layer separated by a gap, said apparatus further comprising means for receiving a first force that constrains motion of said ceiling layer such that said ceiling layer moves independently of said floor layer and means for permitting application of a second force to a control surface inside said lid, wherein a change in said first and second forces initiates a n adjustment of an extent of said gap.

23. The apparatus of claim 22, forming a kit comprising the lid and the device, wherein the device is configured to be supported above the lid in operation, the device comprising:
an actuator including the adjustable member and a motor for causing the adjustable member to change a distance of extension from the device, and in operation to cause a change in the gap between the ceiling layer and the floor layer; and
a plurality of optical sensors configured to acquire optical signals from the probes when in operation the device is contact with the ceiling layer.

24. A method of acquiring signals using the kit of claim 23, the method including:
affixing the lid to the receptacle;
affixing the device on the lid such that the weight of the device is supported by the lid, including passing the adjustable member through the opening in the ceiling layer; and
acquiring signals while oscillating depths of the probes in the wells, including causing the gap to oscillate by operating the motor to cause oscillation in the distance of extension of the adjustable member from the device, and acquiring optical signals from the probes via the optical sensors of the device.

25. An apparatus comprising a lid configured to be disposed between a device and a receptacle with a plurality of wells holding liquid samples such that in operation, the lid is placed on the lid and the device is supported above and in contact with the lid, the lid comprising:
a ceiling layer and a floor layer forming a gap between said layers, wherein
the floor layer is configured to be placed on the receptacle in operation,
the ceiling layer is configured to have contact with the device in operation, and to support at least some of the weight of the device; and
a feature that exerts force between the floor layer and the ceiling layer that opposes closing of the gap, and in operation causes the ceiling layer to maintain contact with the device;
a plurality of elongated probe affixed to the ceiling layer, each passing through a corresponding hole in the floor layer, and in operation protruding below the floor layer into a corresponding well of the receptacle, wherein change in the gap between the floor layer and the ceiling layer causes a change in a length of the probe extending below the floor layer, and in operation causing a change in a depth of the probe in a liquid sample in the well;
wherein the floor layer includes a surface to support in operation a force imparted by an adjustable member of the device extending through an opening in the ceiling layer to the surface without imparting said force on the ceiling layer such that the weight of the device is in part supported by the ceiling layer and in part by said surface, and wherein in operation, adjustment of the adjustable member causes adjustment of the gap and thereby adjustment of the length of the probes extending below the floor layer into the liquid samples in the wells;
wherein the lid provides an air path from an environment of the lid to draw air from the environment into the gap when the gap is increased and to expel air when the gap is decreased, and the floor of the lid provides tortuous paths between the environment and the wells of the receptacle.
